(19) United States
(12) Patent Application Publication
(10) Pub. No.: US 2025/0046398 A1
(43) Pub. Date: Feb. 6, 2025

(54) SYSTEM FOR PREDICTING GENETIC DISEASES AND CONDITIONS USING A NEURAL NETWORK THAT IS TRAINED ON DATA ALIGNED TO A REFERENCE GENOME USING GRAPH ATTENTION MECHANISMS

(71) Applicant: MyOme, Inc., Menlo Park, CA (US)

(72) Inventor: Agust Sverrir Egilsson, Alamo, CA (US)

(21) Appl. No.: 18/798,187

(22) Filed: Aug. 8, 2024

Related U.S. Application Data
(63) Continuation of application No. PCT/US2023/062258, filed on Feb. 9, 2023.
(60) Provisional application No. 63/308,091, filed on Feb. 9, 2022.
(60) Provisional application No. 63/324,539, filed on Mar. 28, 2022.

(51) Int. Cl. G16B 40/20; G16H 50/20

(52) U.S. Cl. CPC G16B 40/20; G16H 50/20

(57) ABSTRACT

Apparatuses, methods, and computer program products are disclosed for training and utilizing a neural network. An example method includes obtaining sequencing variant sample data and training a neural network using the sequencing variant sample data. The example method further includes training the neural network by altering values at one or more loci for one or more samples in the sequencing variant sample data to produce altered sequencing variant sample data and using the altered sequencing variant sample data as input during training of the neural network such that the neural network is trained to predict values from unaltered sequencing variant sample data and the one or more reference genome maps. Another example method includes obtaining subject sequencing variant sample data for a subject and generating predicted values of a genome using a neural network. The method further includes determining one or more predicted conditions for the subject.

### CROSS-REFERENCE TO RELATED APPLICATIONS

This application is a continuation of International Application No. PCT/US2023/062258, filed Feb. 9, 2023, which claims the benefit of U.S. Provisional Application No. 63/308,091 filed on Feb. 9, 2022 and U.S. Provisional Application No. 63/324,536 filed on Mar. 28, 2022, both of which are incorporated herein by reference in their entireties.

### TECHNOLOGICAL FIELD

The present invention relates generally to deep learning based systems that receive genome or multiomics and clinical data input and generate disease classifications or predictions, and more particularly to systems that process genome or multiomics sequences for long regions from one or more chromosomes.

### BACKGROUND

The size of the human genome and the variety of experimental data obtained that maps to positions in the genome is over 3 billion bases long. Even if longer k-mers, i.e., multiple consecutive loci or substrings, are represented as one token, then for a sizable subset of these loci in a deep learning model, a full attention matrix for all these tokens becomes completely impractical due to its size. For example, this may require tera- or petabytes of storage to compute due to its complexity. Attempts to reduce this complexity in the prior art include clustering or focusing the attention mechanism across only a subset of the input, reducing the region being attended to and by reducing the input sequence to a small sequence of loci of interest only.

As a further example the whole genome sequence of a human cancer tissue and a normal tissue as an input into a neural network would require a matrix with approximately 3 billion columns and 10 or 20 rows, if represented in a dense concatenated (e.g., stacked) one-hot encoded format. This is assuming each of the two or four groups of columns specifying the 4 nucleotides (A,C,G,T) and a placeholder for unknown both for the cancer tissue and the normal tissue at all locations and possibly for both pairs of chromosomes for each tissue type. In addition to this, multiple additional dimensions (rows) may track various other genomic features, including but not limited to methylation sites, microsatellite variability, inserts and deletions, and/or the like. Since a network trained using stochastic gradient descent requires multiple such setups batched together, the memory and computational power required get multiplied by order of magnitudes during training. As a result, the solutions found in the prior art, suboptimally, focus on relatively small network input and may require additional filtering of input loci that rely on partially understood knowledge of the underlying biology.

### BRIEF SUMMARY

The embodiments demonstrated show how to input the loci that differ from a given set of reference genome feature mappings, such as multiomics data and nucleotide bases, and at training time to teach the network to merge the reference genome mapping with the incoming data in an indirect, possibly selective, measurable way. In other words, in one or more embodiments, the deep learning system is taught to memorize the reference data and mappings internally. One or more of the embodiments described also teach how to increase the receptive field of the neural network using a stacked/graph neural network structure so that all parts of the input and merged reference data can attend to all other parts of the data during self-supervised pre-training and supervised or semi-supervised fine-tuning during the training processes. This enables, in some embodiments, the network to evaluate the impact of genomic alterations and multiomics across the entire genome in addition to the clinical data included in the embodiments.

### BRIEF DESCRIPTION OF THE DRAWINGS

Having described certain example embodiments in general terms above, reference will now be made to the accompanying drawings, which are not necessarily drawn to scale. Some embodiments may include fewer or more components than those shown in the figures.

FIG. 1 illustrates a high level simplified schematic setup for a training, testing and inference pipeline, which may be used in accordance with some embodiments described herein.

FIG. 2 illustrates a schematic and exemplified neural network graph composed of layers, nodes, functions and transformations between graph levels, which may be used in accordance with some embodiments described herein

FIG. 3 depicts a transformation from one level to another in the neural network graph structure, which may be used in accordance with some embodiments described herein.

FIG. 4 illustrates an example embodiment which injects global metadata (e.g., cell type, gender, cohort cluster, etc.) at the lowest levels in the graphs in order to communicate root level information to the lower graph layers, which may be used in accordance with some embodiments described herein.

FIG. 5 illustrates a flow chart of the pre-training logic that may be used to recreate or memorize the merged reference and input data in a neural network, which may be used in accordance with some embodiments described herein.

FIG. 6 illustrates the use of the root node(s) for classification or regression analysis for exemplified diseases and conditions, which may be used in accordance with some embodiments described herein.

FIG. 7 illustrates an example flowchart for training the neural network to generate one or more predictions, in accordance with some embodiments described herein.

FIG. 8 illustrates an example flowchart for utilizing the neural network to generate one or more predictions for a subject, in accordance with some embodiments described herein.

FIG. 9 illustrates a schematic block diagram of example device that may perform various operations in accordance with some example embodiments described herein.

### DETAILED DESCRIPTION

Some example embodiments will now be described more fully hereinafter with reference to the accompanying figures, in which some, but not necessarily all, embodiments are shown. Because inventions described herein may be embodied in many different forms, the invention should not be limited solely to the embodiments set forth herein; rather, these embodiments are provided so that this disclosure will satisfy applicable legal requirements.

The term “computing device” refers to any one or all of programmable logic controllers (PLCs), programmable automation controllers (PACs), industrial computers, desktop computers, personal data assistants (PDAs), laptop computers, tablet computers, smart books, palm-top computers, personal computers, smartphones, wearable devices (such as headsets, smartwatches, or the like), and similar electronic devices equipped with at least a processor and any other physical components necessarily to perform the various operations described herein. Devices such as smartphones, laptop computers, tablet computers, and wearable devices are generally collectively referred to as mobile devices.

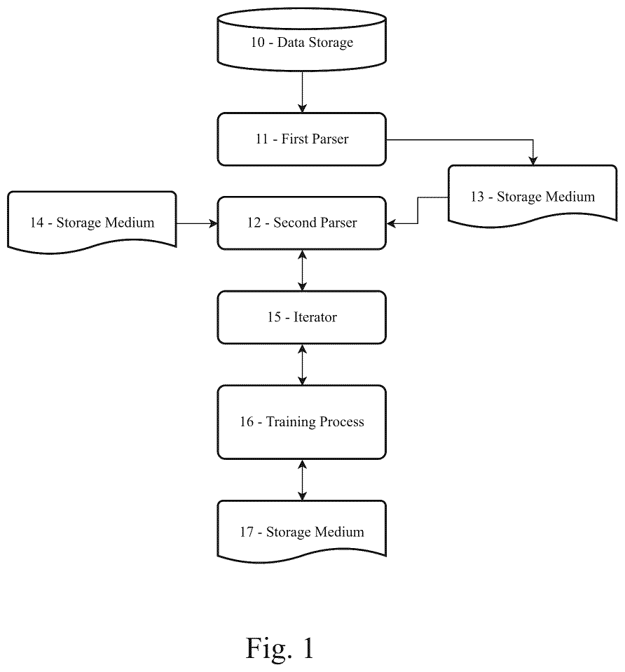

Turning now to FIG. 1, an embodiment of a pipeline required to train a neural network on genome sequencing data and data that maps to genome coordinates (e.g., loci) is illustrated. The elements shown are a tangible, computer-readable medium 10 such as a database or flat file system containing results of sequencing a given tissue as well as, optionally, metadata about the sequencing and each input example and additional multiomics features of each or some loci obtained. The sequencing data may have to be processed by a first parser algorithm 11 and transferred to a second tangible, computer-readable medium or computer memory 13 for efficient processing by the training pipeline shown. The first parser 11 can be instructed to format and store the data as formatted data, such as TensorFlow records or in another efficient format specialized for training neural networks and moving data over networks. A second parser or transform algorithm or both 12 may be used at training time to further process the data and the parsers may have access to one or more reference genomes and other reference data, stored on medium 14. In some embodiments, for example, GRCh38.p13, T2T, or other reference genomes may be used. These reference genomes may allow for a reduction in the size of the genomes or features obtained from data storage 10 or storage medium 13 by selectively eliminating data that is already described by reference data stored by storage medium 14. The reference data may be combined into one or more sets, such as by combining a reference genome with one or more epigenetic maps, such as deoxyribonucleic acid (DNA) methylation maps or other suitable epigenetic maps, in order to match the input feature requirements. Each set may be used to reduce the size of all or a subset of the input cases as determined by other metadata attributes such as gender and cell type. In some embodiments this step is not needed since the genomes and data in data storage 10 already only contain variants and alterations from the reference data.

As part of process performed by the second parser 12, one or more genomes or data aligned to the genome, such as from different samples obtained from tissue or genomic material from the same individual, may be aligned as tensors for input into the network as one example. In some embodiments sparse tensors (e.g., tables with tensor coordinates and values) are used, or other data-types, and placed into a dictionary representing one training example. The said dictionary may also contain multiomics data, originating from data storage 10, which may also be read and processed by first parser 11. The multiomics data may include data such as methylation sites found in the respective tissue or genetic material, chromatin accessibility information related to or experimentally obtained for the tissue, and/or the like. In cases when a model is being fine-tuned or otherwise trained in a supervised way, labels may be extracted from the data storages (e.g., data storage 10 and/or storage medium 13) as well. For pre-training, some embodiments use a masked training schema, as has become common in the field when training large foundation models, such as bidirectional encoder representations (BERT) models. In some embodiments, masking is achieved by selecting random slices of a limited size from the genome and optimizing the model (e.g., using the associated weights) to predict the merged reference and input sequences in specific layers at the masked sequence locations (e.g., loci). Self-supervised pre-training such as masking is also, in some embodiments, assigned other optimization tasks, including predicting genomic alterations, chromatin accessibility sites, epigenomic features, somatic or germline variant locations, and/or the like. In order to achieve this, a set of labels and masks is collected as part of process performed by second parser 12 and these labels and masks are added to the dictionary or sparse tensor before multiple examples are batched together for a training step.

During training, an iterator algorithm 15 may be used to loop over multiple epochs of the training data and may divide the data into a training data set, validation data set, and testing data set. In some embodiments the training process 16 optimizes the self-supervised pre-training tasks. In some embodiments, the training process 16 optimizes the neural network using supervised learning based on labels such as disease type, phenotypes or epigenomic features. Some embodiments optimize for both concurrently. Some embodiments store the trained weights one or more times on a storage medium 17, both after and during training based on set conditions, such as early stopping. Before training starts, some embodiments may load existing weights from previous training session as initial weights instead of randomizing the initial values of the weights following standard deep learning approaches.

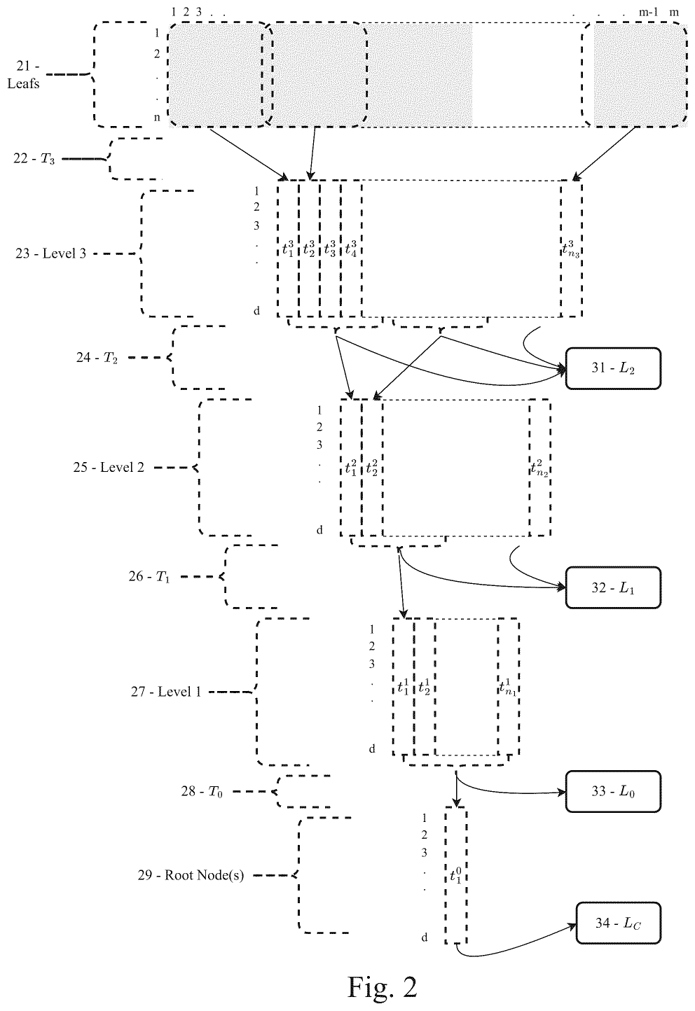

FIG. 2 shows a schematic and exemplified neural network graph composed of layers, which may be used in some embodiments, such as for the analysis of a large dataset, like multiomics data mapped to genome coordinates/loci. In particular, FIG. 2 depicts a graph structure of stacked transformers, similar to those used in BERT models and/or other deep learning kernels. In one example, the input data into the network for one input case or example is shown as matrix at leaf level 21, which may include one or more stacked one-hot encoded matrices aligned to the human genome coordinates, indicating deviations from the reference data in storage medium 14 for each of the stacked matrices. The matrix may contain mostly zeros and such that it may be stored efficiently as a sparse matrix representation with most or all zeros removed. For example, the matrix may be stored as a list or lists of pairs of matrix coordinates and nonzero or mostly nonzero matrix values as well as other metadata such as the dense tensor or matrix shape. This can be done because of the well understood nature of one-hot encoded data as well as the sparsity of variations in individuals from the reference genome in humans and other species in general.

In some embodiments, most or all the genome data that aligns with the reference values in storage medium 14 is simply represented by zero valued columns and is therefore ignored by the sparse matrix representation of the matrix. In other embodiments a non-zero encoding is used to represent reference data. In some embodiments the sparsity is such that the input matrix is reduced based on the variants found in each individual case itself, in some other embodiments a standard set of variant positions can be used. So even if in an example where the size of the matrix is n rows and m columns, with m being greater than 3 billion, in some embodiments, the sparse matrix representation is efficiently stored as a list (e.g., sparse matrix). The same is true in other embodiments for smaller matrices, for example when restricting to the exome sequence only or a subset of genes or a combination of sequences of exon and intron regions as well as sequences from the noncoding regions, in these cases the matrix structure is equally efficiently stored as a sparse matrix of variant data only.

The data in the matrix in leaf level 21 is mapped to individual tokens in the level 3 grouping 23. Each token is denoted as tₙ^{L}, where n is the indexed position of the token within the grouping and L is the corresponding level of the token within the graph or hierarchy. In some embodiments, the level 3 grouping 23 is a collection of nodes corresponding to tokens 1 through n₃, where n₃ is the total number of tokens included at the third level. Each token may be a vector of fixed dimension d, which may be selected as appropriate for level in the graph or hierarchy. In some embodiments the transformation function 22 is a trainable linear map that maps a sparse submatrix starting from column 1 and stretching to column k (k being a fixed number selected for said embodiment) to the first d dimensions of the first token in the level 3 grouping 23 before moving to repeat the process for subsequence columns in the matrix. In some embodiments the linear mapping has stride length less than one full step of said k columns so that subsequent tokens are created from overlapping entries in the matrix. In some embodiments multiple distinct trainable linear maps are used producing multiple tokens representing said columns 1 to k. The embedding dimension shown as d for the level 3 grouping 23, level 2 grouping 25, level 1 grouping 27, and root node grouping 29 may be varied between layers, as determined by hyperparameter optimization or other suitable selection processes.

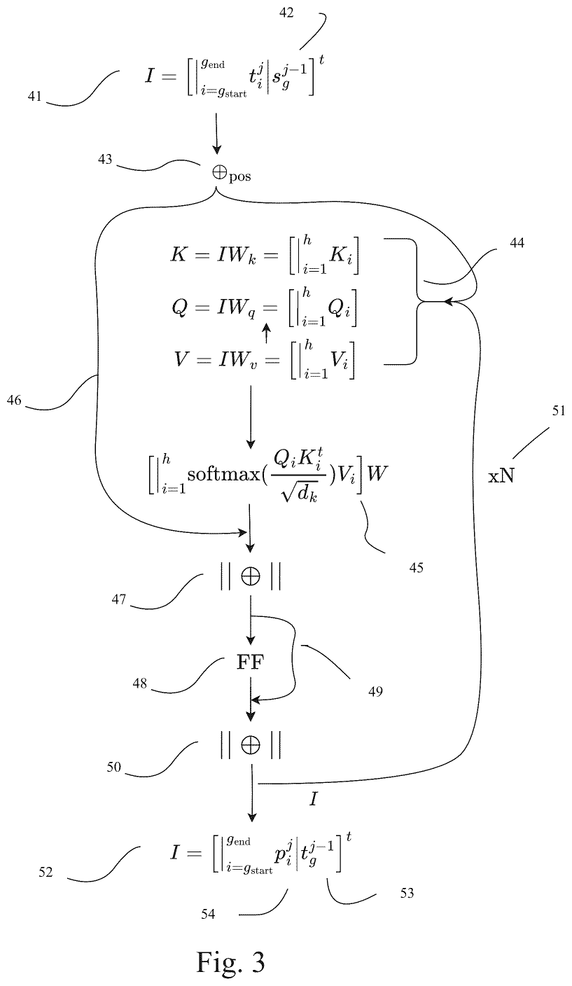

For illustrative purposes, one can set m in the level 3 grouping 21 to be 3 billion, n to be 10, k (e.g., the number of columns for a sparse submatrix) to be 1000, d in the level 3 grouping 23 to 1000, and the stride length to be 1000 such that no overlap is produced. In that example the number of tokens produced in the level 3 grouping 23 is 3 million and each token is of length (dimensionality) 1000. In this illustrative setup, each token/embedding of dimension 1000 now represents 1000 consecutive bases in the genome even if only variations from the reference data in storage medium 14 are included at this first stage. The tokens are shown as nodes at the third level grouping 23 in the graph. During processing by the training algorithm, the matrices have various shapes as determined for computational purposes using well known tensor reshape methods available in neural network training frameworks, such as TensorFlow. An additional batch dimension, not shown but considered, may also be included at training time thereby allowing multiple examples forming a mini-batch of cases to be included in one step of the optimization. In some embodiments non-linear maps may be used in transformation function 22 instead of said trainable linear map including transformer based maps, such as the one shown in FIG. 3.

In some embodiments the tokens at level 3 are assigned trainable or fixed positional encodings (e.g., see FIG. 3 positional encoding operation 43 for additional details) such that each token can represent the genomic interval in the previous level (e.g., leaf level 21) moving forward. The tokens at level 3 are subdivided into groups of consecutive tokens and each group g of tokens passed through a shared, in some embodiments, for that level, trainable multi head attention mechanism (e.g., a transformer encoder) illustrated further in FIG. 3. As further described in FIG. 3, additional tokens are created to represent the progression, through the attention mechanism, of one or more outputs or parent node tokens at the next level and are assigned positional encodings which are included in said multihead attention mechanism.

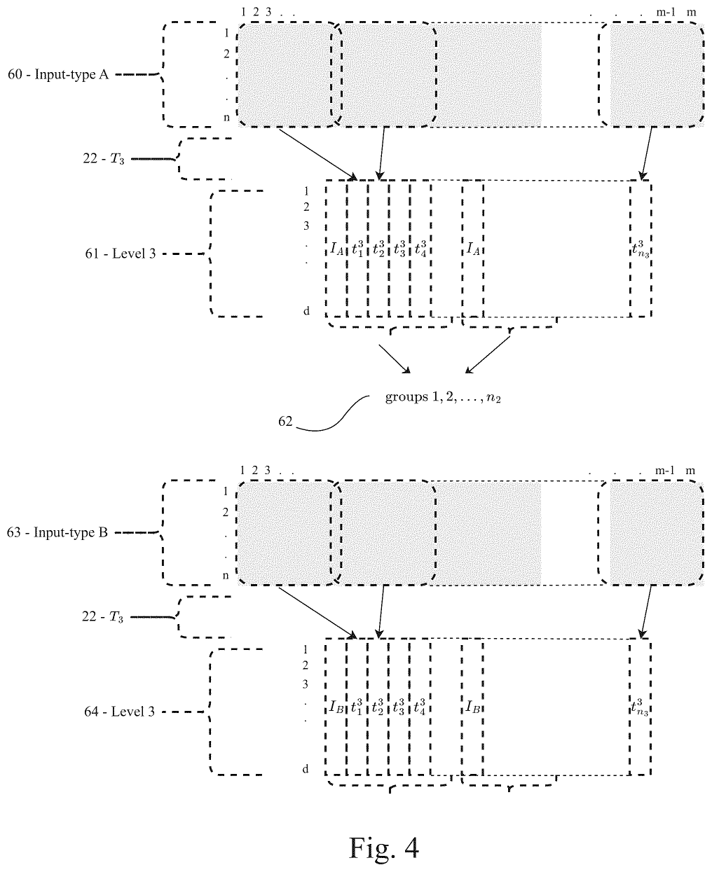

In some embodiments one or more additional input-type tokens are added, and shared, per group of tokens as trainable or fixed tokens to distinguish between different input cases such as a token representing the input cell, tissue type, gender, ancestry, or other covariates or cohort groups as explained further by FIG. 4. In cases when more than one parent token node is represented, some embodiments concatenate (e.g., via a concat function) the resulting parent node tokens before placing them at the next level (in the direction of the root node) in the graph. Additionally, the embedding dimension d may be increased accordingly at said next level to accommodate the increased length of the embeddings. It will be noted that FIG. 2 and in FIG. 3 show only one additional token for simplicity. However, it will be appreciated by one of skill in the art that one or more additional tokens may be added to represent a parent token node. The one or more additional tokens may then be processed by the transformer encoder at the corresponding level and then returned as a representative of the group as one or more parent token nodes in the graph at the coarser next level (e.g., in the direction of the root node on FIG. 2 and in accordance with operations described in FIG. 3).

Focusing on the first group in the level 3 grouping 23, one or more additional tokens initialized with positional encodings are added to the first group and then all the tokens from this first group and the additional tokens (e.g., both the additional output (parent node) tokens and the input-type tokens) are mapped by the transformer encoder 24 (e.g., T₂). The mappings resulting from the additional said output (e.g., parent node) tokens are transferred to the next level being constructed, e.g., level 2 grouping 25 as parent nodes for the child nodes in the first group in the level 3 grouping 23 in the graph since these represent the complete group.

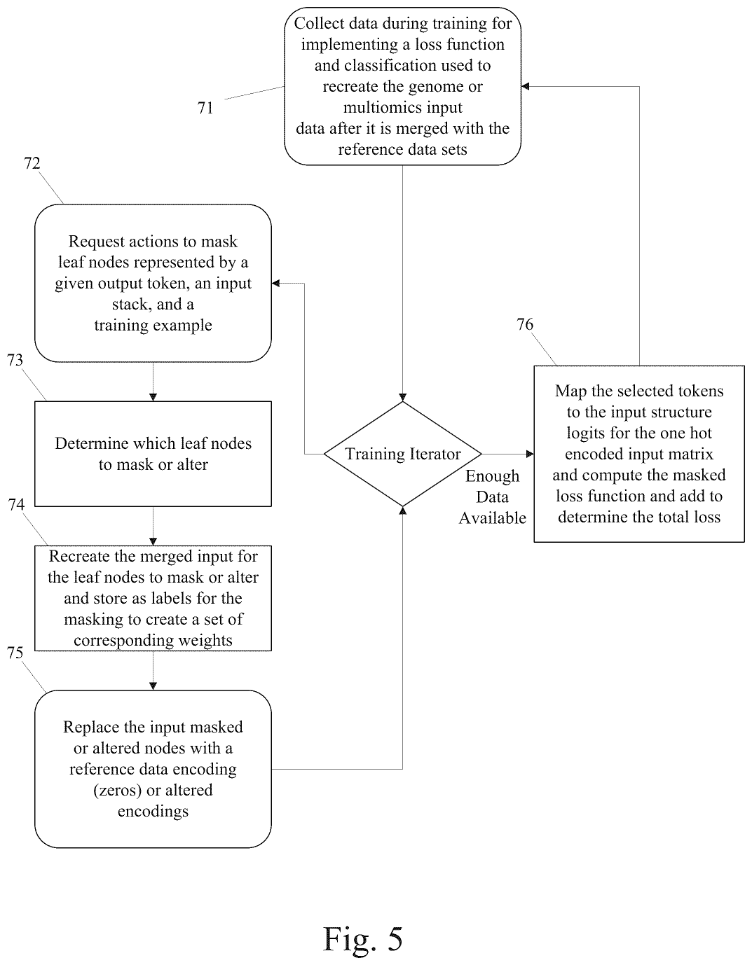

In some embodiments, the results (e.g., output 54 in FIG. 3) of applying the multihead attention mechanism to the group are processed further and used to create loss functions 31 for the layer that then is used along with other loss contributions to optimize the layer and the neural network (weights) to predict, using masked training schema, the reference data, labels or genome (e.g., reference data stored by storage medium 14 merged with the input data from matrix). In some embodiments the loss function 31 is used to recreate the merged incoming genotypes after the reference genome has been merged with the variants. In other embodiments the multiomics data after merging the reference data is also predicted using the loss function. In some embodiments the self-supervised masked training implemented by the loss function 31 are implemented according to the operations described in FIG. 5. Training this loss function teaches the network to present to the next level through the multi head self-attention mechanism, selectively, the data that was excluded as reference data from the input and gives the neural network the ability to work with the merged input data and the reference data moving forward in the direction of the root node grouping 29. Thereby, through this and other pre-training, in some embodiments, a large network learns to memorize the merged representation of the sparse input data and the reference data.

To exemplify the transformer encoder 24 shown in FIG. 2, assuming again for illustration, that only the first 1000 tokens are included in the first group then an additional token, initialized with positional encodings only, is created and a total of 1001 tokens are passed through the shared transformer encoder 24 used for the transformation. The 1001 vectors resulting from the process are then divided into two parts: (i) the results of the original 1000 tokens after being processed by the transformer, which are used to establish a loss function able to predict, internally in the network the merged incoming and reference data (as opposed to inputting this data) and (ii) the last 1001-th token, which moves forward as an embedding for the group representing the span of the genome covered by the group. At each iteration only a small fraction of the 1000 tokens, which randomly and iteratively selected, are used to determine the loss value of said loss function. In some embodiments, this fraction may range between 15 percent to 20 percent of the tokens.

The above process of grouping tokens together and passing the grouped tokens through transformer kernels (e.g., transformer encoder 24 and transformer encoder 26 (e.g., T₁)) is repeated as many times as needed until the number of tokens left or the height of the graph (e.g., the number of levels) has reached the desired limit. In some embodiments, as shown on FIG. 2, the process is repeated using independent multihead self-attention one or two more times, such as using transformer encoder 26 and transformer encoder 28 (e.g., T₀)). This results in tokens in the level 1 grouping 27 and a final single token t₁⁰ in the root node grouping 29. This process is further shown and described in FIG. 3. The final embedding is then further processed through transformations as a classification/regression prediction output into an overall objective loss function such as a function predicting the likelihood of a number of disease conditions, cancer staging, cancer classification or predicting other quantities such as normalized expected expression values or attributes of the data, such as cell type using the loss function 34. As such, the network is then optimized to predict one or more conditions during training. In some embodiments, the loss function 32 and loss function 33 are assigned tasks that include predicting alterations at the loci corresponding to various tokens and used as loss contributions during self-supervised training. In some embodiments the last transformation by transformation encoder 28 to the root node grouping 29 in the diagram is simply a linear projection starting from the last token from the processed tokens at the previous level since self-attention mechanisms may not be necessary when working with one token only.

FIG. 3 is referenced several times in the description above and is described in more detail herein. FIG. 3 depicts an example of an encoder from a transform network which may be used in some embodiments. The encoder described in FIG. 3 may be similar to transformation encoders commonly used and available in BERT models. Although FIG. 3 depicts one particular implementation of a transformation encoder, it will be appreciated that these transformation encoders may include variations such as additional layer normalization, dropout, activations, regularizers and other additional methods and network layers.

As shown in FIG. 3, a given group g 41 of tokens is taken as input and includes one or more additional tokens 42 in addition to the tokens within the group. These tokens are assigned trainable or fixed positional encodings using encoding operation 43. FIG. 3 then shows elements from the standard transformation encoder designs, such as key K, query Q, and value V matrices 44 being formed in each iteration and divided up into h heads. The K, Q, V matrices 44 are then used by the multihead self-attention mechanism 45. The first residual connection 46 and the layer normalization 47 are provided to a feed forward network 48. A residual connection 49 is also shown with an additional layer normalization 50. The steps are repeated N times as indicated by operation 51 and the final output 52 shows embeddings indicated by p variables 54 that are used in predictions and the parent token t_{g}^{j-1} 53. Parent token 53 is the next level token in the graph representing the parent node of the tokens 41. The hyperparameters used to set the sizes (e.g. trainable weights) and behavior (e.g. dropout rate) of each of the elements in the network shown are not necessarily identical between levels. In some embodiments the hyperparameters are tuned individually for each of the levels in the graph.

FIG. 4 exemplifies the use of learned input-type tokens to communicate the input type to the lowest grouping level in the neural network graph structure. The top matrix shows an input-type A matrix 60 enumerated as input type “A”. In some embodiments this input type corresponds to a specific cell type, tissue type, an exome sequence, a whole genome sequence, or to identify a species “A” or any other metadata associated with the input example. The token shown as “I_{A}” is added to each of the groups 62 at the lowest grouping level 61 (e.g., level 3 grouping). A different input type matrix 63 would be issued a different input-type token shown as “I_{B}” at the lowest grouping level 64, assuming it belongs to a different input type (e.g., a different cell type, tissue type, exome sequence, whole genome sequence, or species).

In some embodiments the input-type tokens “I_{A}”, “I_{B}”, etc. are d-dimensional trainable vectors. In some embodiments, these input-type tokens are constant vectors. In some embodiments multiple input-type tokens are used per input to represent multiple input attributes. For example, one input-type token may be used to represent the cell type and another input-type token may be used at the same time to represent the gender. Encoding the input-type representation in this way communicates root level (e.g., example-based) information to the lowest grouping level and allows the network to perform input-type specific computation at each of the grouping levels in cases when such information is assumed or shown to improve the network's performance and is available at inference time. In some embodiments the input type tokens represent grouping of the input data based on clustering, such as clustering representing different ancestry cohorts. This may be the case when the input data can be significantly reduced by having different reference genomes or reference data for each of the clusters. In some embodiments the input embeddings represent continuous variables such as scores from various other databases and algorithms. For example, the input embedding may include aggregated REVEL scores for specific regions, EVE scores for genes and other variables used to measure the pathogenicity or loss of function of proteins and DNA sequences, or available polygenic risk scores (PRS) for the individual being analyzed or screened with respect to a given disease. In some embodiments continuous variables are mapped by binning the range, (e.g., from zero to one), such that different bins are represented by different embeddings which are either learned or fixed. In some embodiments, continuous variables are embedded directly using linear transformations starting with a set of one or more such values.

FIG. 5 shows a high level flow chart of the part of the training process used by some embodiments to learn, during pre-training or fine-tuning, the merged genome or multiomics data for individual training cases. The training described in FIG. 5 allows a neural network to learn the merged genome and/or multiomics data even for instances where only the data that differs from the reference data is the input to the neural network. At the same time, the described masking optimization challenges the network to learn the frequency and likelihood of the functional genomics presented to the network as part of the input data through the masking and alterations of the input. In the context at hand, part of the input is masked both from the variants data (e.g., the variants for a particular stack from the matrix as well as the reference data from the storage medium 14). In particular, operation 71 collects data during training in order to add a loss contribution to the neural network training that forces the network to recreate internally the merged genome or multiomics input data after it is merged with the reference data from storage medium 14. This is done by masking a subset from the input as well as changing some of the input values or reference values and having the network predict the masked or correct values using corresponding loss functions, such as loss function 31.

In some embodiments, this is done by a neural network training iterator that calls a subprocess operations 72, 73, 74 and 75. At subprocess operation 72, the neural network training iterator requests actions to mask one or more leaf nodes represented by a given output token, an input stack, and a training example. In some embodiments generate or are supplied one or more random token graph positions (e.g., from level 3 grouping 23 in the case of loss function 31), that identify a subset of leaf nodes from matrix.

At subprocess operation 73, the neural network training iterator determines which leaf nodes to mask and/or alter. In some embodiments, the neural network training iterator randomly selects leaf nodes to mask or alter (e.g., 15% of said subset of leaf nodes). In some embodiments the input leaf nodes are one or more stacked one-hot encoded matrices, and the masking is directed toward one or more of these stacked matrices.

At subprocess operation 74, the neural network training iterator recreates the merged input for the leaf nodes to mask or alter and stores as labels for the masking to create a set of corresponding weights. Since the masked loss requires training labels, these training labels are generated. In some embodiments, these training labels are generated by procedurally recreating the merged input data and the reference data for the stacked matrix selected. The labels may then be stored in memory, or otherwise, before the corresponding input data for a given training case is temporarily removed from the input matrix for one training round (e.g., epoch).

At subprocess operation 75, the neural network training iterator may replace the input masked or altered nodes with a reference data encoding (e.g., zeros) or altered encodings.

In some embodiments, in addition to masking a fraction (e.g., 5%) of the leaf nodes from the leaf node subset, a fraction of the leaf nodes are instead altered, either by replacing a one-hot encoding by a different altered one-hot encoding or keeping it unchanged and still have the loss function (e.g., loss function 31), predict the correct class encoding during classification. The masking may require one or more weight matrices to be created and stored for use as additional input to the loss function, in order to direct the loss function to only consider predictions for said randomly selected leaf nodes.

At operation 76, once enough data is available, the training iterator may map selected tokens to input structure logits for the one hot encoded matrix and compute the masked loss function and add to determine the total loss. In some embodiments a mini-batch of masked examples is repeatedly created and once a batch is ready for processing the loss function is applied using logits directly, or a softmax function to normalize the encoding predictions. In some embodiments, the predictions (logits) are generated by a trainable and shared linear map that maps the final forward pass tokens encoded from said token graph positions (e.g. from level 3 grouping 23 in case of loss function 31, by the respective transformer encoder 24) to a matrix structure matching the selected leaf nodes from the stacked matrix selected as part of the input. The loss function (e.g., loss function 31) then compares the predictions by the network, using said linear map, with the respective stored labels generated earlier (e.g., at operation 74) and using the stored loss weights. In some embodiments a classification cross-entropy loss function is used for this purpose and added with appropriate weight to the total loss for the network. In some embodiments, one or more of the loss functions at higher levels, such as (e.g., loss function 32 and/or loss function 33) are used analogously, using respectively higher level tokens and encoders to recreate masked or altered leaf nodes internally.

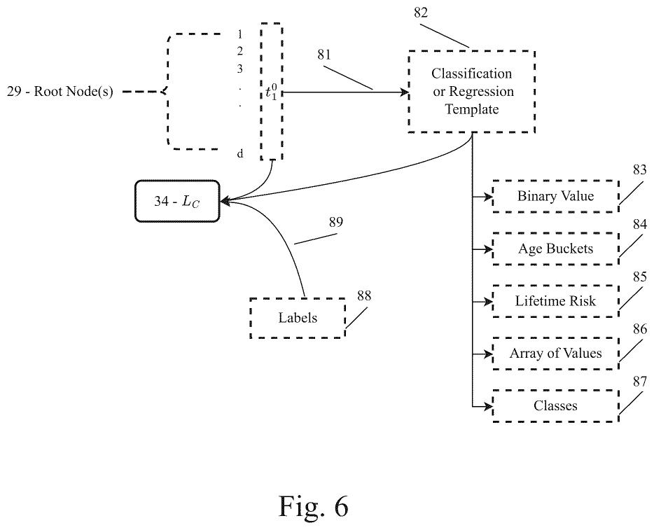

FIG. 6 illustrates the use of root nodes for classification and/or regression analysis for exemplified diseases and conditions. In particular, FIG. 6 shows a mapping operation 81 of one or more root tokens from root node grouping 29 to a structure matching the desired classification or regression template, for training and inference. In some embodiments said template 82 is an array of values 86. In some embodiments, template 82 includes logits or normalized likelihoods (e.g., softmax values) or a single value representing binary values 83. In some embodiments, template 82 includes one or more other values resulting from a forward pass of the input data through the neural network exemplified in FIG. 2.

The classification or regression template is used to train the neural network (e.g., using loss function 34) during fine-tuning of the network or during both training phases, fine-tuning and pre-training. The classification or regression labels 88 used during the training are available during training and may be extracted from the data storage 10 and/or data storage 13 for each or some of the training examples. In some embodiments the labels 88 are processed at operation 89 during training to match said template and/or inject noise into the training.

For clarification, an example of such processing is the mapping of the labels to the correct age buckets representing the onset or continuation of a disease condition. In some embodiments the classification template is composed of one or more age buckets 84, each receiving a likelihood prediction representative of the likelihood of a disease or medical condition for the patient being analyzed or screened. In some embodiments when predicting the (polygenic) likelihood of breast cancer from genetic and functional data obtained by sequencing and other methods, the age buckets 84 are used as templates to score the likelihood of the condition for the patient in each of the age ranges defined by the age buckets. Similarly, in some embodiments, including when determining cardiovascular disease risk scores, risk of developing schizophrenia or type one or two diabetes, age buckets 86 or lifetime risk 85 likelihood variables are used to predict the risk to the individual.

In some embodiments the input to the neural network includes variants from whole exome sequencing or whole genome sequencing and the input may be restricted to genomic regions containing genes and other sequences assumed to be involved in the development of the disease. Other phenotypes may be predicted using the appropriate categorical classes. In some embodiments when predicting categorical values a cross-entropy loss function is used as the loss measure by loss function 34 and when predicting the output in multiple buckets (e.g., age buckets) the template generates multiple binary scores per training example. In some embodiments the processing during operation 89 generates loss weights for each bucket. The loss weights are used, in some embodiments, to exclude buckets where no information is available about the disease condition of an individual in a given age range. Additionally, loss weights may be used to address biases and balance datasets to represent a particular technique associated with oversampling or undersampling. In some embodiments cell expression (e.g., mRNA) measurements, and other quantitative traits may be predicted using the array of values 86 template, matching the available labels. In some embodiments when predicting quantitative traits, a Poisson or least square loss function is used as the loss function measure for loss function 34.

Some embodiments use human exome sequencing data as input. These embodiments may map a small number of genome positions (e.g. 1 to 128) from the matrix to each individual token at the third level group 23 as described in connection with FIG. 2. In some embodiments composed genotypes, phased or unphased are one hot encoded and homozygous genotypes matching the reference genome are treated as reference genotypes, in this case only variants from the reference need then to be included in the input sparse matrix representation of matrix. Some embodiments only include single nucleotide polymorphisms while others also one-hot encode inserts and deletions as variants. Some embodiments include larger regions containing the exome data as input in order to capture, for example, splicing variants while some embodiments include large sections of the non-coding regions which may contain regulatory regions. The labels used by some embodiments include onset of diseases such as breast cancer, schizophrenia, cardiovascular disease, type one and two diabetes and other diseases with a genetic component from large cohorts of participants in genetic research. Some embodiments also construct labels using the disease phenotype and a combination of other attributes such as the age of onset or being unaffected by the disease or condition (e.g., negative or control samples).

Some embodiments use multiple additional techniques well known in the art such as additional linear layers, normalizations, convolutions, dropout, tokenizers, specific input layers, activation functions, weight initialization, weight regularizers, momentum, different type of optimizers, variations of stochastic gradient descent and different type of one-hot encoding schemas. Other embodiments of the invention such as applying the teaching to a sizable fraction of the genome or exome instead of the complete genome should be considered as being within the scope of the invention.

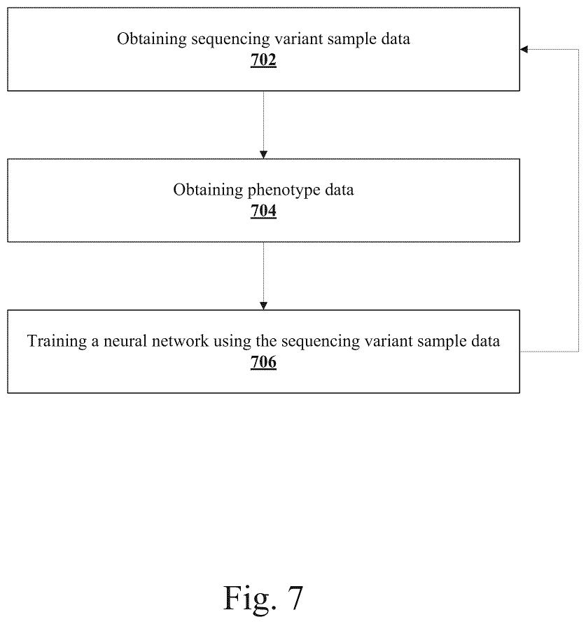

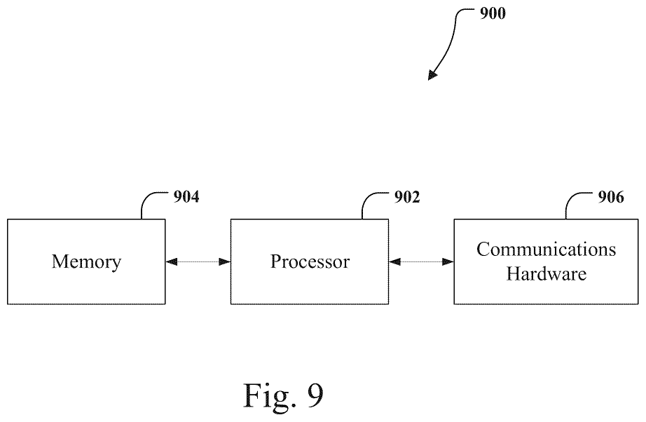

Turning to FIG. 7, an example flowchart is illustrated that contains example operations implemented by various embodiments contemplated herein. In particular, FIG. 7 depicts example operations that may be performed to train a neural network to generate one or more predictions. The operations illustrated in FIG. 7 may, for example, be performed by an apparatus 900, which is shown and described in connection with FIG. 9. To perform the operations described below, the apparatus 900 may utilize one or more of processor 902, memory 904, communications hardware 906, other components, and/or any combination thereof. It will be understood that user interaction with the apparatus 900 may occur directly via input-output circuitry (not shown), or may instead be facilitated by a device that in turn interacts with apparatus 900.

As shown by operation 702, the apparatus 900 includes means, such as processor 902, memory 904, communications hardware 906, or the like, for obtaining sequencing variant sample data. The sequencing variant sample data may be based on a sequencing sample dataset and one or more reference genome maps. In some embodiments, the sequencing variant sample data is obtained similarly to the above processes/operations described in FIG. 1. As described above, the sequencing variant sample data may include data describing sequencing data and/or multiomics features for loci obtained from one or more samples for a corresponding subject. In some embodiments, a subject may refer to an entity such as a person, cell type, cancer type, or other biological entity. A sample may be any biological matter that may be sequenced including but not limited to tissue samples, blood samples, fluid samples, cell-specific samples (e.g., samples taken from a malignant tumor), and/or the like. In some embodiments, the sample identity and/or sequencing performed may be described as associated sequencing variant sample metadata.

In some embodiments, the sequencing variant sample data may be obtained directly from storage, such as from storage medium 13, and may not require further processing. In some embodiments, the sequencing variant sample data may include variants obtained from whole genome sequencing or whole exome sequencing. The particular variants obtained may depend on whether a corresponding sample was sequenced using a whole genome sequencing process or a whole exome sequencing process. In some embodiments, the sequencing variant sample data is one-hot encoded, as described above with reference to FIG. 1.

In some embodiments, the sequencing variant sample data may be obtained via generation of the sequencing variant sample data. In particular, apparatus 900 may receive reference data and formatted sequencing sample data. In some embodiments, reference data may be received by identifying one or more reference datasets (e.g., as stored in storage medium 14) and generating combined reference data by combining the one or more reference datasets with one or more epigenetic maps. Here, the one or more epigenetic maps may include DNA methylation maps. The combined reference data may then be received by apparatus 900 such that is then used as the reference data. In some embodiments, the formatted sequencing sample data may be received by accessing raw sequencing sample data (e.g., stored in data storage 10) and formatting the raw sequencing sample data to produce formatted raw sequencing sample data. For example, the raw sequencing sample data may be stored in data storage 10 and may then be formatted by the first parser 11 to a suitable format (e.g., TensorFlow reports) for later processing by the neural network. The formatted raw sequencing sample data may then be received by apparatus 900 and used as the formatted sequencing sample data. Obtaining the sequencing variant sample data is described in more detail above with reference to FIG. 1.

Apparatus 900 may then identify values shared between the reference data and the formatted sequencing sample data. Once these shared values have been identified, apparatus 900 may remove the one or more values shared between the reference data and the formatted sequencing sample data to generate the sequencing variant sample data. As such, apparatus 900 may selectively eliminate data already described by the reference data.

As shown by operation 704, the apparatus 900 includes means, such as processor 902, memory 904, communications hardware 906, or the like, for obtaining phenotype data. The phenotype data may include disease onset labels and/or control labels for one or more of conditions which are known for a corresponding subject. For example, the one or more conditions may include breast cancer, cardiovascular disease, type one diabetes, type two diabetes, schizophrenia, or a combination thereof which are associated with the sample data. In some embodiments, the phenotype data may also describe an associated PRS score for a known condition of the subject. The phenotype data may be stored with the sequencing variant sample data and/or raw sequencing sample data, such as in data storage 10 and/or storage medium 13.

As shown by operation 706, the apparatus 900 includes means, such as processor 902, memory 904, communications hardware 906, or the like, for training the neural network using the sequencing variant sample data. In some embodiments, the apparatus 900 may store the neural network and corresponding attributes (e.g., weights, loss functions, hyperparameters, etc.) in an associated memory, such as memory 904, storage medium 17, or a remote data storage. Apparatus 900 may access the stored neural network in an instance a training program is initiated and/or received by apparatus 900. As described above, a neural network may be trained to generate one or more predictions for a subject and further, may be trained using the sequencing variant sample data as obtained in operation 802 as input for the neural network. The neural network may be trained to predict values from unaltered sequencing sample data and one or more reference genome maps. The training of the neural network may include altering values at one or more loci for one or more samples in the sequencing variant sample data to produce altered sequencing variant sample data and using the altered values for training of the neural network. Additionally, the one or more layers of the neural network may be trained to predict phenotype data associated with the sequencing variant sample data. As described in further detail with respect to FIG. 6, classification or regression labels may be constructed based on phenotype data such that the neural network may be trained using labeled phenotype data for the sequencing variant sample data.

In particular, the neural network may be trained by training one or more layers of the neural network by performing one or more training iterations. As described above and shown in FIG. 2, the neural network may include one or more layers arranged into a hierarchy of attention encoder mechanisms. Each level in the hierarchy is then subdivided into one or more groups (e.g., submatrices). Then for each group, one or more parent tokens and in some embodiments, additional tokens, are input into the attention encoder mechanism associated with the particular group and used as an input token for attention encoder mechanisms as a subsequent level. Here, the graph structure of the neural network moves in the direction towards the root node level, as shown and described further in FIG. 2.

In some embodiments, an input-type token is appended to the one or more groups. As described above, an input-type token may correspond to one or more of a cell type, a tissue type, an exome sequence, a whole genome sequence, or a species. These input-type tokens may be appended to the lowest grouping level in the neural network.

During each training iteration, one or more actual values of the sequencing variant sample data from the one or more loci and one or more samples corresponding to the sequencing variant sample data may be altered. This altered sequencing variant sample data may be used as input for the neural network such that the neural network is trained to predict values from unaltered sequencing variant sample data and one or more reference genome maps. The neural network may then generate one or more predicted values for these one or more loci and the one or more samples, and the neural network may be adjusted based on the one or more predicted values and the one or more actual values. In particular, the loss weights of one or more loss functions in the neural network may be adjusted in accordance with the operations described in FIG. 6. In some embodiments, the operation of altering the values may be performed by masking a fraction of the values of the sequencing variant sample data (e.g., 10 to 15 percent of the sequencing variant sample data). In some embodiments, the operation of altering the values may be performed by masking a fraction of the values of the sequencing variant sample data (e.g., 10 to 15 percent of the sequencing variant sample data) and replacing a fraction of the values of the sequencing variant sample data (e.g., 5 to 10 percent of the sequencing variant sample data).

The above described operations described in FIG. 7 may be repeated any number of times to train or retrain the neural network. In some embodiments, subsequent trainings of the neural network may result in updated attributes (e.g., weight, hyperparameters, etc.) of the neural network. These updated attributes and the neural network model may be logged and saved by apparatus 900 in a corresponding memory, such as memory 904. In an instance the model is to be trained and/or retrained, apparatus 900 may be configured to query and/or access the most recent neural network and attributes such that the most up-to-date model is used.

Additionally, although operation 7 describes operations 702 through 706 as performed by apparatus 900, it will be appreciated that each operation may be performed by a separate and/or remote apparatus. For example, operation 702 for obtaining sequencing variant sample data may be performed by an apparatus A while operation 706 for training the neural network may be performed by an apparatus B. Additionally, data storage 10, storage medium 13, storage medium 14, and/or storage medium 17 of FIG. 1 and as described above in FIG. 7 may each be separate and/or remote storage medium or may be a shared storage.

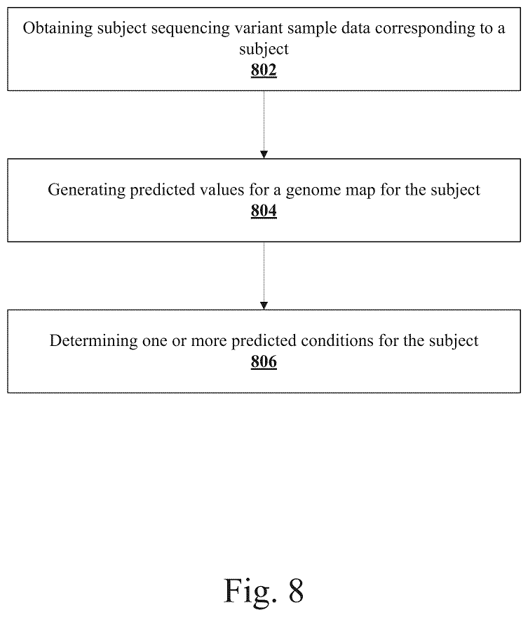

Turning now to FIG. 8, an example flowchart is illustrated that contains example operations implemented by various embodiments contemplated herein. The operations illustrated in FIG. 8 may, for example, be performed by an apparatus 900, which is shown and described in connection with FIG. 9. To perform the operations described below, the apparatus 900 may utilize one or more of processor 902, memory 904, communications hardware 906, other components, and/or any combination thereof. It will be understood that user interaction with the apparatus 900 may occur directly via input-output circuitry (not shown), or may instead be facilitated by a device that in turn interacts with apparatus 900.

As shown by operation 802, the apparatus 900 includes means, such as processor 902, memory 904, communications hardware 906, or the like, for obtaining subject sequencing variant sample data corresponding to a subject. As described above in operation 702, a subject may refer to an entity such as a person, cell type, cancer type, or other biological entity. In particular, obtaining subject sequencing variant sample data may include sequencing a sample from the subject to produce raw sequenced sample data. Sequenced data may be obtained from the subject, such as via whole genome sequencing or whole exome sequencing of a sample from the subject. A sample may be any biological matter that may be sequenced including but not limited to tissue samples, blood samples, fluid samples, cell-specific samples (e.g., samples taken from a malignant tumor), and/or the like. The raw sample data may be formatted (e.g., to a TensorFlow record) to produce the sequencing sample data for the subject.

Once the sample data is obtained, apparatus 900 may receive reference data. Similarly, as described above for operation 702, reference data may be received by identifying one or more reference datasets (e.g., as stored in storage medium 14) and generating combined reference data by combining the one or more reference datasets with one or more epigenetic maps. Here, the one or more epigenetic maps may include DNA methylation maps. The combined reference data may be received by apparatus 900 such that is then used as the reference data. Apparatus 900 may then identify values shared between the reference data and the sample data for the subject. Apparatus 900 may then remove one or more values shared between the reference data and the sample data to yield the subject sequencing variant sample data for the subject.

As shown by operation 804, the apparatus 900 includes means, such as processor 902, memory 904, communications hardware 906, or the like, for generating predicted values for a genome map for the subject. In particular, apparatus 900 may input the subject sequencing variant sample data into a neural network which has been trained, such as via the operations described above in FIG. 7. Apparatus 900 may access and utilize the neural network from an associated storage, such as memory 904, storage medium 17, or a remote data storage. The neural network may be configured to predict values of a genome map for the subject based on the input sequencing variant sample data. As such, although only variant data was provided as input to the neural network, the neural network may be configured to infer the values for the genome map.

As shown by operation 806, the apparatus 900 includes means, such as processor 902, memory 904, communications hardware 906, or the like, for generating one or more predicted conditions for the subject. In some embodiments, the neural network may also be configured to generate one or more predicted conditions for the subject based on the predicted values. For example, as described above in greater detail in FIG. 6, the neural network may be trained using classification or regression templates such that it is able to generate one or more predicted conditions by scoring the likelihood of the condition for each subject. In particular, in an instance a subject is determined to have a score that satisfies a score threshold for a particular condition, the neural network may predict said condition for the subject. In some embodiments, the neural network may be configured to generate a predicted polygenic risk score for a predicted condition for the subject. The neural network may generate a predicted polygenic risk score for a condition based on the one or more predicted risk scores for said condition.

In some embodiments, the neural network may generate and output the one or more predictions and/or predicted values for the genome map for the subject as a report, which may be sent to one or more computing devices, such as computing devices of clinical practitioners, geneticists, researchers, patients, and/or the like. In some embodiments, the neural network may generate and output the one or more predictions and/or predicted values for the genome map for the subject as input to other downstream models.

### Example Implementation System

The methods described herein may be implemented on a variety of systems. For instance, in some embodiments the system may be used for obtaining sequencing variant sample data, obtaining phenotype data, training a neural network, obtaining subject sequencing variant sample data, generating predictions for a genome map for a subject, determining one or more predicted conditions for a subject, etc. The below described apparatus 900 may be used to perform any combination of these operations. In some embodiments, one or more of the above described apparatuses, such as the training iterator described in FIG. 5, may be implemented as apparatus 900.

The system may include one or more system devices, which may be embodied by one or more computing devices or servers, shown as apparatus 900 in FIG. 9. As illustrated in FIG. 9, the apparatus 900 may include a processor 902, memory 904, and communications hardware 906, each of which will be described in greater detail below. While the various components are only illustrated in FIG. 9 as being connected with apparatus 900, it will be understood that the apparatus 900 may further comprise a bus (not expressly shown in FIG. 9) for passing information amongst any combination of the various components of the apparatus 900. The apparatus 900 may be configured to execute various operations described above.

The processor 902 (and/or co-processor or any other processor assisting or otherwise associated with the processor) may be in communication with the memory 904 via a bus for passing information amongst components of the apparatus. The processor 902 may be embodied in a number of different ways and may, for example, include one or more processing devices configured to perform independently. Furthermore, the processor may include one or more processors configured in tandem via a bus to enable independent execution of software instructions, pipelining, and/or multithreading. The use of the term “processor” may be understood to include a single core processor, a multi-core processor, multiple processors of the apparatus 900, remote or “cloud” processors, or any combination thereof.

The processor 902 may be configured to execute software instructions stored in the memory 904 or otherwise accessible to the processor (e.g., software instructions stored on a separate storage device). In some cases, the processor may be configured to execute hard-coded functionality. As such, whether configured by hardware or software methods, or by a combination of hardware with software, the processor 902 represents an entity (e.g., physically embodied in circuitry) capable of performing operations according to various embodiments of the present invention while configured accordingly. Alternatively, as another example, when the processor 902 is embodied as an executor of software instructions, the software instructions may specifically configure the processor 902 to perform the algorithms and/or operations described herein when the software instructions are executed.

The memory 904 is non-transitory and may include, for example, one or more volatile and/or non-volatile memories. In other words, for example, the memory 904 may be an electronic storage device (e.g., a computer readable storage medium). The memory 904 may be configured to store information, data, content, applications, software instructions, or the like, for enabling the apparatus to carry out various functions in accordance with example embodiments contemplated herein. Additionally, data storage 10, storage medium 13, storage medium 14, and/or storage medium 17 of FIG. 1 may be embodied by memory 904. Data storage 10, storage medium 13, storage medium 14, and/or storage medium 17 may each be separate and/or remote storage mediums or may be a shared storage.

The communications hardware 906 may be any means such as a device or circuitry embodied in either hardware or a combination of hardware and software that is configured to receive and/or transmit data from/to a network and/or any other device, circuitry, or module in communication with the apparatus 900. In this regard, the communications hardware 906 may include, for example, a network interface for enabling communications with a wired or wireless communication network. For example, the communications hardware 906 may include one or more network interface cards, antennas, buses, switches, routers, modems, and supporting hardware and/or software, or any other device suitable for enabling communications via a network. Furthermore, the communications hardware 906 may include the processing circuitry for causing transmission of such signals to a network or for handling receipt of signals received from a network.

The communications hardware 906 may be configured to provide output to a user and, in some embodiments, to receive an indication of user input. The communications hardware 906 comprises a user interface, such as a display, and may further comprise the components that govern use of the user interface, such as a web browser, mobile application, dedicated user device, or the like. In some embodiments, the communications hardware 906 may include a keyboard, a mouse, a touch screen, touch areas, soft keys, a microphone, a speaker, and/or other input/output mechanisms. The communications hardware 906 may utilize the processor 902 to control one or more functions of one or more of these user interface elements through software instructions (e.g., application software and/or system software, such as firmware) stored on a memory (e.g., memory 904) accessible to the processor 902.

### CONCLUSION

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

Clause 1. A method comprising: obtaining sequencing variant sample data, wherein the sequencing variant sample data is based on sequencing sample data and one or more reference genome maps; and training a neural network using the sequencing variant sample data, wherein training the neural network includes: altering values at one or more loci for one or more samples in the sequencing variant sample data to produce altered sequencing variant sample data, and using the altered sequencing variant sample data as input during training of the neural network such that the neural network is trained to predict values from unaltered sequencing variant sample data and the one or more reference genome maps.

Clause 2. The method of clause 1, wherein training the neural network comprises training one or more layers of the neural network by performing one or more training iterations, each training iteration including: altering one or more actual values of the sequencing variant sample data from the one or more loci and the one or more samples corresponding to the sequencing variant sample data; generating, using the neural network, one or more predicted values for the one or more loci and the one or more samples, and adjusting the neural network based on the one or more predicted values and the one or more actual values.

Clause 3. The method of any of clauses 1 to 2, further comprising: obtaining phenotype data associated with the sequencing variant sample data; and training one or more layers of the neural network to predict the phenotype data.

Clause 4. The method of any of clauses 1 to 3, wherein (i) the one or more layers are arranged into a hierarchy of attention encoder mechanisms, (ii) a level in the hierarchy is subdivided into one or more groups and (iii) for each group, using one or more parent tokens from another attention encoder mechanism associated with the group as an input token for an attention encoder mechanism at a subsequent level in the hierarchy.

Clause 5. The method of any of clauses 1 to 4, further comprising appending an input-type token to the one or more groups.

Clause 6. The method of any of clauses 1 to 5, wherein the input-type token corresponds to an input type and the input type corresponds to one or more of a cell type, a tissue type, an exome sequence, a whole genome sequence, or a species.

Clause 7. The method of any of clauses 1 to 6, wherein the sequencing variant sample data comprises variants obtained from whole exome sequencing.

Clause 8. The method of any of clauses 1 to 7, wherein the sequencing variant sample data comprises variants obtained from whole genome sequencing.

Clause 9. The method of any of clauses 1 to 8, wherein the phenotype data includes disease onset and control labels for one or more conditions.

Clause 10. The method of any of clauses 1 to 9, wherein the one or more conditions comprise breast cancer, cardiovascular disease, type one diabetes, type two diabetes, schizophrenia, or a combination thereof.

Clause 11. The method of any of clauses 1 to 10, wherein altering the values further comprises masking a fraction of the values of the sequencing variant sample data.

Clause 12. The method of any of clauses 1 to 11, wherein altering the values further comprises masking a first fraction of the values of the sequencing variant sample data and replacing a second fraction of the values of the sequencing variant sample data.

Clause 13. The method of any of clauses 1 to 12, wherein the sequencing variant sample data is one-hot encoded.

Clause 14. The method of any of clauses 1 to 13, wherein obtaining the sequencing variant sample data further comprises: receiving reference data and formatted sequencing sample data; identifying one or more values shared between the reference data and the formatted sequencing sample data; and removing one or more of the one or more values shared between the reference data and the formatted sequencing sample data.

Clause 15. The method of any of clauses 1 to 14, wherein receiving the reference data further comprises: identifying one or more reference datasets; and generating combined reference data by combining the one or more reference datasets with one or more epigenetic maps, wherein the reference data comprises the combined reference data.

Clause 16. The method of any of clauses 1 to 15, wherein the one or more epigenetic maps comprises one or more deoxyribonucleic acid methylation maps.

Clause 17. The method of any of clauses 1 to 16, wherein receiving the formatted sequencing sample data further comprises: accessing raw sequencing sample data; and formatting the raw sequencing sample data to produce formatted raw sequencing sample data, wherein the formatted sequencing sample data is obtained using the formatted raw sequencing sample data to produce the sequencing variant sample data.

Clause 18. The method of any of clauses 1 to 17, further comprising: obtaining subject sequencing variant sample data, wherein the subject sequencing variant sample data corresponds to sequenced sample data for a subject; and generating, using the neural network, predicted values of a genome map for the subject based on the subject sequencing variant sample data.

Clause 19. The method of any of clauses 1 to 18, further comprising determining one or more predicted conditions for the subject.

Clause 20. The method of any of clauses 1 to 19, wherein the one or more predicted conditions comprise breast cancer, cardiovascular disease, type one diabetes, type two diabetes, schizophrenia, or a combination thereof.

Clause 21. The method of any of clauses 1 to 20, wherein the one or more predicted conditions comprise a polygenic risk score determined for a particular condition for the subject, wherein one or more predicted risk scores for the particular condition are used to determine the polygenic risk score for the particular condition.

Clause 22. The method of any of clauses 1 to 21, further comprising: sequencing a sample from the subject to produce raw sequenced sample data; and formatting the raw sequenced sample data to produce the sequenced sample data for the subject.

Clause 23. The method of any of clauses 1 to 22, further comprising: receiving reference data; identifying one or more values shared between the reference data and the sample data for the subject; and removing one or more of the one or more values shared between the reference data and the formatted sequencing sample data to yield the subject sequencing variant sample data for the subject.

Clause 24. The method of any of clauses 1 to 23, wherein the sample is sequenced using one or more of whole exome sequencing or whole genome sequencing.

Clause 25. An apparatus comprising a processor and a memory storing software instructions that, when executed by the processor, cause the apparatus to: obtain sequencing variant sample data, wherein the sequencing variant sample data is based on sequencing sample data and one or more reference genome maps; and train a neural network using the sequencing variant sample data, wherein training the neural network includes: altering values at one or more loci for one or more samples in the sequencing variant sample data to produce altered sequencing variant sample data to produce altered sequencing variant sample data, and using the altered sequencing variant sample data as input during training of the neural network such that the neural network is trained to predict values from unaltered sequencing variant sample data and the one or more reference genome maps.

Clause 26. The apparatus of clause 25, wherein the software instructions, when executed by the processor and when training the neural network, further cause the apparatus to train one or more layers of the neural network by performing one or more training iterations, wherein each performing each training iteration further causes the apparatus to: alter one or more actual values of the sequencing variant sample data from the one or more loci and the one or more samples corresponding to the sequencing variant sample data; generate, using the neural network, one or more predicted values for the one or more loci and the one or more samples, and adjusting the neural network based on the one or more predicted values and the one or more actual values.

Clause 27. The apparatus of any of clauses 25 to 26, wherein the software instructions, when executed by the processor, further cause the apparatus to obtain phenotype data associated with the sequencing variant sample data; and training one or more layers of the neural network to predict the phenotype data.

Clause 28. The apparatus of any of clauses 25 to 27, wherein (i) the one or more layers are arranged into a hierarchy of attention encoder mechanisms, (ii) a level in the hierarchy is subdivided into one or more groups and (iii) for each group, using one or more parent tokens from another attention encoder mechanism associated with the group as an input token for an attention encoder mechanism at a subsequent level in the hierarchy.

Clause 29. The apparatus of any of clauses 25 to 28, wherein the software instructions, when executed by the processor, further cause the apparatus to append an input-type token to the one or more groups.

Clause 30. The apparatus of any of clauses 25 to 29, wherein the input-type token corresponds to an input type and the input type corresponds to one or more of a cell type, a tissue type, an exome sequence, a whole genome sequence, or a species.

Clause 31. The apparatus of any of clauses 25 to 30, wherein the sequencing variant sample data comprises variants obtained from whole exome sequencing.

Clause 32. The apparatus of any of clauses 25 to 31, wherein the sequencing variant sample data comprises variants obtained from whole genome sequencing.

Clause 33. The apparatus of any of clauses 25 to 32 wherein the phenotype data includes disease onset and control labels for one or more conditions.

Clause 34. The apparatus of any of clauses 25 to 33, wherein the one or more conditions comprise breast cancer, cardiovascular disease, type one diabetes, type two diabetes, schizophrenia, or a combination thereof.

Clause 35. The apparatus of any of clauses 25 to 34, wherein the software instructions, when executed by the processor and when altering the values, further cause the apparatus to mask a fraction of the values of the sequencing variant sample data.

Clause 36. The apparatus of any of clauses 25 to 35, wherein the software instructions, when executed by the processor and when altering the values, further cause the apparatus to mask a first fraction of the values of the sequencing variant sample data and replace a second fraction of the values of the sequencing variant sample data.

Clause 37. The apparatus of any of clauses 25 to 36, wherein the sequencing variant sample data is one-hot encoded.

Clause 38. The apparatus of any of clauses 25 to 37, wherein the software instructions, when executed by the processor and when obtaining the sequencing variant sample data, further cause the apparatus to: receive reference data and formatted sequencing sample data; identify one or more values shared between the reference data and the formatted sequencing sample data; and remove one or more of the one or more values shared between the reference data and the formatted sequencing sample data.

Clause 39. The apparatus of any of clauses 25 to 38, wherein the software instructions, when executed by the processor and when receiving the reference data, further cause the apparatus to: identify one or more reference datasets; and generate combined reference data by combining the one or more reference datasets with one or more epigenetic maps, wherein the reference data comprises the combined reference data.

Clause 40. The apparatus of any of clauses 25 to 39, wherein the one or more epigenetic maps comprises one or more deoxyribonucleic acid methylation maps.

Clause 41. The apparatus of any of clauses 25 to 40, wherein the software instructions, when executed by the processor and when receiving the formatted sequencing sample data, further cause the apparatus to access raw sequencing sample data; and format the raw sequencing sample data to produce formatted raw sequencing sample data, wherein the formatted sequencing sample data is obtained using the formatted raw sequencing sample data to produce the sequencing variant sample data.

Clause 42. The apparatus of any of clauses 25 to 41, wherein the software instructions, when executed by the processor and obtaining the sequencing variant sample data, further cause the apparatus to: obtain subject sequencing variant sample data, wherein the subject sequencing variant sample data corresponds to sequenced sample data for a subject; and generate, using the neural network, predicted values of a genome map for the subject based on the subject sequencing variant sample data.

Clause 43. The apparatus of any of clauses 25 to 42, wherein the software instructions, when executed by the processor and obtaining the sequencing variant sample data, further cause the apparatus to determine one or more predicted conditions for the subject.

Clause 44. The apparatus of any of clauses 25 to 43, wherein the one or more predicted conditions comprise breast cancer, cardiovascular disease, type one diabetes, type two diabetes, schizophrenia, or a combination thereof.

Clause 45. The apparatus of any of clauses 25 to 44, wherein the one or more predicted conditions comprise a polygenic risk score determined for a particular condition for the subject, wherein one or more predicted risk scores for the particular condition are used to determine the polygenic risk score for the particular condition.

Clause 46. The apparatus of any of clauses 25 to 45, wherein the software instructions, when executed by the processor and when obtaining the sequencing variant sample data, further cause the apparatus to sequence a sample from the subject to produce raw sequenced sample data; and format the raw sequenced sample data to produce the sequenced sample data for the subject.

Clause 47. The apparatus of any of clauses 25 to 46, wherein the software instructions, when executed by the processor and when obtaining the sequencing variant sample data, further cause the apparatus to receive reference data; identify one or more values shared between the reference data and the sample data for the subject; and remove one or more of the one or more values shared between the reference data and the formatted sequencing sample data to yield the subject sequencing variant sample data for the subject.

Clause 48. The apparatus of any of clauses 25 to 47, wherein the sample is sequenced using one or more of whole exome sequencing or whole genome sequencing.

Clause 49. A computer program product comprising at least one non-transitory computer-readable storage medium storing software instructions that, when executed by an apparatus, cause the apparatus to: obtain sequencing variant sample data, wherein the sequencing variant sample data is based on sequencing sample data and one or more reference genome maps; and train a neural network using the sequencing variant sample data, wherein training the neural network includes: altering values at one or more loci for one or more samples in the sequencing variant sample data to produce altered sequencing variant sample data to produce altered sequencing variant sample data, and using the altered sequencing variant sample data as input during training of the neural network such that the neural network is trained to predict values from unaltered sequencing variant sample data and the one or more reference genome maps.

Clause 50. The computer program product of clause 49, wherein the software instructions, when executed by the processor and for training the neural network, further cause the apparatus to train one or more layers of the neural network by performing one or more training iterations, wherein each performing each training iteration further causes the apparatus to: alter one or more actual values of the sequencing variant sample data from the one or more loci and the one or more samples corresponding to the sequencing variant sample data; generate, using the neural network, one or more predicted values for the one or more loci and the one or more samples, and adjusting the neural network based on the one or more predicted values and the one or more actual values.

Clause 51. The computer program product of any of clauses 49 to 50, wherein the software instructions, when executed by the processor, further cause the apparatus to obtain phenotype data associated with the sequencing variant sample data; and training one or more layers of the neural network to predict the phenotype data.

Clause 52. The apparatus of any of clauses 49 to 51, wherein (i) the one or more layers are arranged into a hierarchy of attention encoder mechanisms, (ii) a level in the hierarchy is subdivided into one or more groups and (iii) for each group, using one or more parent tokens from another attention encoder mechanism associated with the group as an input token for an attention encoder mechanism at a subsequent level in the hierarchy.

Clause 53. The computer program product of any of clauses 49 to 52, wherein the software instructions, when executed by the processor, further cause the apparatus to append an input-type token to the one or more groups.

Clause 54. The computer program product of any of clauses 49 to 53, wherein the input-type token corresponds to an input type and the input type corresponds to one or more of a cell type, a tissue type, an exome sequence, a whole genome sequence, or a species.

Clause 55. The computer program product of any of clauses 49 to 54, wherein the sequencing variant sample data comprises variants obtained from whole exome sequencing.

Clause 56. The computer program product of any of clauses 49 to 55, wherein the sequencing variant sample data comprises variants obtained from whole genome sequencing.

Clause 57. The computer program product of any of clauses 49 to 56 wherein the phenotype data includes disease onset and control labels for one or more conditions.

Clause 58. The computer program product of any of clauses 49 to 57, wherein the one or more conditions comprise breast cancer, cardiovascular disease, type one diabetes, type two diabetes, schizophrenia, or a combination thereof.

Clause 59. The computer program product of any of clauses 49 to 58, wherein the software instructions, when executed by the processor and when altering the values, further cause the apparatus to mask a fraction of the values of the sequencing variant sample data.

Clause 60. The computer program product of any of clauses 49 to 59, wherein the software instructions, when executed by the processor and when altering the values, further cause the apparatus to mask a first fraction of the values of the sequencing variant sample data and replace a second fraction of the values of the sequencing variant sample data.

Clause 61. The computer program product of any of clauses 49 to 60, wherein the sequencing variant sample data is one-hot encoded.

Clause 62. The computer program product of any of clauses 49 to 61, wherein the software instructions, when executed by the processor and when obtaining the sequencing variant sample data, further cause the apparatus to: receive reference data and formatted sequencing sample data; identify one or more values shared between the reference data and the formatted sequencing sample data; and remove one or more of the one or more values shared between the reference data and the formatted sequencing sample data.

Clause 63. The computer program product of any of clauses 49 to 62, wherein the software instructions, when executed by the processor and when receiving the reference data, further cause the apparatus to: identify one or more reference datasets; and generate combined reference data by combining the one or more reference datasets with one or more epigenetic maps, wherein the reference data comprises the combined reference data.

Clause 64. The computer program product of any of clauses 49 to 63, wherein the one or more epigenetic maps comprises one or more deoxyribonucleic acid methylation maps.

Clause 65. The computer program product of any of clauses 49 to 64, wherein the software instructions, when executed by the processor and when receiving the formatted sequencing sample data, further cause the apparatus to access raw sequencing sample data; and format the raw sequencing sample data to produce formatted raw sequencing sample data, wherein the formatted sequencing sample data is obtained using the formatted raw sequencing sample data to produce the sequencing variant sample data.

Clause 66. The computer program product of any of clauses 49 to 65, wherein the software instructions, when executed by the processor and obtaining the sequencing variant sample data, further cause the apparatus to: obtain subject sequencing variant sample data, wherein the subject sequencing variant sample data corresponds to sequenced sample data for a subject; and generate, using the neural network, predicted values of a genome map for the subject based on the subject sequencing variant sample data.

Clause 67. The computer program product of any of clauses 49 to 66, wherein the software instructions, when executed by the processor and obtaining the sequencing variant sample data, further cause the apparatus to determine one or more predicted conditions for the subject.

Clause 68. The computer program product of any of clauses 49 to 67, wherein the one or more predicted conditions comprise breast cancer, cardiovascular disease, type one diabetes, type two diabetes, schizophrenia, or a combination thereof.

Clause 69. The computer program product of any of clauses 49 to 68, wherein the one or more predicted conditions comprise a polygenic risk score determined for a particular condition for the subject, wherein one or more predicted risk scores for the particular condition are used to determine the polygenic risk score for the particular condition.

Clause 70. The computer program product of any of clauses 49 to 69, wherein the software instructions, when executed by the processor and when obtaining the sequencing variant sample data, further cause the apparatus to sequence a sample from the subject to produce raw sequenced sample data; and format the raw sequenced sample data to produce the sequenced sample data for the subject.

Clause 71. The computer program product of any of clauses 49 to 70, wherein the software instructions, when executed by the processor and when obtaining the sequencing variant sample data, further cause the apparatus to receive reference data; identify one or more values shared between the reference data and the sample data for the subject; and remove one or more of the one or more values shared between the reference data and the formatted sequencing sample data to yield the subject sequencing variant sample data for the subject.

Clause 72. The computer program product of any of clauses 49 to 71, wherein the sample is sequenced using one or more of whole exome sequencing or whole genome sequencing.

Clause 73. A method comprising: obtaining subject sequencing variant sample data, wherein the subject sequencing variant sample data corresponds to sequenced sample data for a subject; generating, using a neural network trained to predict values of a genome map created from sample data and one or more reference genome maps, predicted values of a genome map for the subject based on the subject sequencing variant sample data; and determining, based on the predicted values, one or more predicted conditions for the subject.

Clause 74. The method of clause 73, further comprising: sequencing a sample from the subject to produce raw sequenced sample data; and formatting the raw sequenced sample data to produce the sequenced sample data for the subject.

Clause 75. The method any of clauses 73 to 74, further comprising: receiving reference data; identifying one or more values shared between the reference data and the sample data for the subject; and removing one or more of the one or more values shared between the reference data and the formatted sequencing sample data to yield the subject sequencing variant sample data for the subject.

Clause 76. The method of any of clauses 73 to 75, wherein the sample is sequenced using one or more of whole exome sequencing or whole genome sequencing.

Clause 77. The method of any of clauses 73 to 76, wherein the one or more predicted conditions comprise breast cancer, cardiovascular disease, type one diabetes, type two diabetes, schizophrenia, or a combination thereof.

Clause 78. The method of any of clauses 73 to 77, wherein the one or more predicted conditions comprise a polygenic risk score determined for a particular condition for the subject, wherein one or more predicted risk scores for the particular condition are used to determine the polygenic risk score for the particular condition.

Clause 79. The method of any of clauses 73 to 78, further comprising: obtaining sequencing variant sample data, wherein the sequencing variant sample data is based on sequencing sample data and one or more reference genome maps; and training a neural network using the sequencing variant sample data, wherein training the neural network includes: altering values at one or more loci for one or more samples in the sequencing variant sample data to produce altered sequencing variant sample data, and using the altered sequencing variant sample data as input during training of the neural network such that the neural network is trained to predict values from unaltered sequencing variant sample data and the one or more reference genome maps.

Clause 80. The method of any of clauses 73 to 79, wherein training the neural network comprises training one or more layers of the neural network by performing one or more training iterations, each training iteration including: altering one or more actual values of the sequencing variant sample data from the one or more loci and the one or more samples corresponding to the sequencing variant sample data; generating, using the neural network, one or more predicted values for the one or more loci and the one or more samples, and adjusting the neural network based on the one or more predicted values and the one or more actual values.

Clause 81. The method of any of clauses 73 to 80, further comprising: obtaining phenotype data associated with the sequencing variant sample data; and training one or more layers of the neural network to predict the phenotype data.

Clause 82. The method of any of clauses 73 to 81, wherein (i) the one or more layers are arranged into a hierarchy of attention encoder mechanisms, (ii) a level in the hierarchy is subdivided into one or more groups and (iii) for each group, using one or more parent tokens from another attention encoder mechanism associated with the group as an input token for an attention encoder mechanism at a subsequent level in the hierarchy.

Clause 83. The method of any of clauses 73 to 82, further comprising appending an input-type token to the one or more groups.

Clause 84. The method of any of clauses 73 to 83, wherein the input-type token corresponds to an input type and the input type corresponds to one or more of a cell type, a tissue type, an exome sequence, a whole genome sequence, or a species.

Clause 85. The method of any of clauses 73 to 84, wherein the sequencing variant sample data comprises variants obtained from whole exome sequencing.

Clause 86. The method of any of clauses 73 to 85, wherein the sequencing variant sample data comprises variants obtained from whole genome sequencing.

Clause 87. The method of any of clauses 73 to 86, wherein the phenotype data includes disease onset and control labels for one or more conditions.

Clause 88. The method of any of clauses 73 to 87, wherein the one or more conditions comprise breast cancer, cardiovascular disease, type one diabetes, type two diabetes, schizophrenia, or a combination thereof.

Clause 89. The method of any of clauses 73 to 88, wherein altering the values further comprises masking a fraction of the values of the sequencing variant sample data.

Clause 90. The method of any of clauses 73 to 89, wherein altering the values further comprises masking a first fraction of the values of the sequencing variant sample data and replacing a second fraction of the values of the sequencing variant sample data.

Clause 91. The method of any of clauses 73 to 90, wherein the sequencing variant sample data is one-hot encoded.

Clause 92. The method of any of clauses 73 to 91, wherein obtaining the sequencing variant sample data further comprises: receiving reference data and formatted sequencing sample data; identifying one or more values shared between the reference data and the formatted sequencing sample data; and removing one or more of the one or more values shared between the reference data and the formatted sequencing sample data.

Clause 93. The method of any of clauses 73 to 92, wherein receiving the reference data further comprises: identifying one or more reference datasets; and generating combined reference data by combining the one or more reference datasets with one or more epigenetic maps, wherein the reference data comprises the combined reference data.

Clause 94. The method of any of clauses 73 to 93, wherein the one or more epigenetic maps comprises one or more deoxyribonucleic acid methylation maps.

Clause 95. The method of any of clauses 73 to 94, wherein receiving the formatted sequencing sample data further comprises: accessing raw sequencing sample data; and formatting the raw sequencing sample data to produce formatted raw sequencing sample data, wherein the formatted sequencing sample data is obtained using the formatted raw sequencing sample data to produce the sequencing variant sample data.

Clause 96. An apparatus comprising a processor and a memory storing software instructions that, when executed by the processor, cause the apparatus to: obtain subject sequencing variant sample data, wherein the subject sequencing variant sample data corresponds to sequenced sample data for a subject; generate, using a neural network trained to predict values of a genome map created from sample data and one or more reference genome maps, predicted values of a genome map for the subject based on the subject sequencing variant sample data; and determine, based on the predicted values, one or more predicted conditions for the subject.

Clause 97. The apparatus of clause 96, wherein the memory storing software instructions, when executed by the processor, further cause the apparatus to sequence a sample from the subject to produce raw sequenced sample data; and formatting the raw sequenced sample data to produce the sequenced sample data for the subject.

Clause 98. The apparatus of any of clauses 96 to 97, wherein the memory storing software instructions, when executed by the processor, further cause the apparatus to: receive reference data; identifying one or more values shared between the reference data and the sample data for the subject; and removing one or more of the one or more values shared between the reference data and the formatted sequencing sample data to yield the subject sequencing variant sample data for the subject.

Clause 99. The apparatus of any of clauses 96 to 98, wherein the sample is sequenced using one or more of whole exome sequencing or whole genome sequencing.

Clause 100. The apparatus of any of clauses 96 to 99, wherein the one or more predicted conditions comprise breast cancer, cardiovascular disease, type one diabetes, type two diabetes, schizophrenia, or a combination thereof.

Clause 101. The apparatus of any of clauses 96 to 100, wherein the one or more predicted conditions comprise a polygenic risk score determined for a particular condition for the subject, wherein one or more predicted risk scores for the particular condition are used to determine the polygenic risk score for the particular condition.

Clause 102. The apparatus of any of clauses 96 to 101, wherein the memory storing software instructions, when executed by the processor, further cause the apparatus to: obtain sequencing variant sample data, wherein the sequencing variant sample data is based on sequencing sample data and one or more reference genome maps; and train a neural network using the sequencing variant sample data, wherein training the neural network includes: altering values at one or more loci for one or more samples in the sequencing variant sample data to produce altered sequencing variant sample data, and using the altered sequencing variant sample data as input during training of the neural network such that the neural network is trained to predict values from unaltered sequencing variant sample data and the one or more reference genome maps.

Clause 103. The apparatus of any of clauses 96 to 102, wherein the software instructions, when executed by the processor and when training the neural network, further cause the apparatus to train one or more layers of the neural network by performing one or more training iterations, wherein each performing each training iteration further causes the apparatus to: alter one or more actual values of the sequencing variant sample data from the one or more loci and the one or more samples corresponding to the sequencing variant sample data; generate, using the neural network, one or more predicted values for the one or more loci and the one or more samples, and adjusting the neural network based on the one or more predicted values and the one or more actual values.

Clause 104. The apparatus of any of clauses 96 to 103, wherein the software instructions, when executed by the processor, further cause the apparatus to obtain phenotype data associated with the sequencing variant sample data; and train one or more layers of the neural network to predict the phenotype data.

Clause 105. The apparatus of any of clauses 96 to 104, wherein (i) the one or more layers are arranged into a hierarchy of attention encoder mechanisms, (ii) a level in the hierarchy is subdivided into one or more groups and (iii) for each group, using one or more parent tokens from another attention encoder mechanism associated with the group as an input token for an attention encoder mechanism at a subsequent level in the hierarchy.

Clause 106. The apparatus of any of clauses 96 to 105, wherein the software instructions, when executed by the processor, further cause the apparatus to append an input-type token to the one or more groups.

Clause 107. The apparatus of any of clauses 96 to 106, wherein the input-type token corresponds to an input type and the input type corresponds to one or more of a cell type, a tissue type, an exome sequence, a whole genome sequence, or a species.

Clause 108. The apparatus of any of clauses 96 to 107, wherein the sequencing variant sample data comprises variants obtained from whole exome sequencing.

Clause 109. The apparatus of any of clauses 96 to 108, wherein the sequencing variant sample data comprises variants obtained from whole genome sequencing.

Clause 110. The apparatus of any of clauses 96 to 109, wherein the phenotype data includes disease onset and control labels for one or more conditions.

Clause 111. The apparatus of any of clauses 96 to 110, wherein the one or more conditions comprise breast cancer, cardiovascular disease, type one diabetes, type two diabetes, schizophrenia, or a combination thereof.

Clause 112. The apparatus of any of clauses 96 to 111, wherein the software instructions, when executed by the processor and when altering the values, further cause the apparatus to mask a fraction of the values of the sequencing variant sample data.

Clause 113. The apparatus of any of clauses 96 to 112, wherein the software instructions, when executed by the processor and when altering the values, further cause the apparatus to mask a first fraction of the values of the sequencing variant sample data and replace a second fraction of the values of the sequencing variant sample data.

Clause 114. The apparatus of any of clauses 96 to 113, wherein the sequencing variant sample data is one-hot encoded.

Clause 115. The apparatus of any of clauses 96 to 114, wherein the software instructions, when executed by the processor and when obtaining the sequencing variant sample data, further cause the apparatus to: receive reference data and formatted sequencing sample data; identify one or more values shared between the reference data and the formatted sequencing sample data; and remove one or more of the one or more values shared between the reference data and the formatted sequencing sample data.

Clause 116. The apparatus of any of clauses 96 to 115, wherein the software instructions, when executed by the processor and when receiving the reference data, further cause the apparatus to: identify one or more reference datasets; and generate combined reference data by combining the one or more reference datasets with one or more epigenetic maps, wherein the reference data comprises the combined reference data.

Clause 117. The apparatus of any of clauses 96 to 116, wherein the one or more epigenetic maps comprises one or more deoxyribonucleic acid methylation maps.

Clause 118. The apparatus of any of clauses 96 to 117, wherein the software instructions, when executed by the processor and when receiving the formatted sequencing sample data, further cause the apparatus to access raw sequencing sample data; and format the raw sequencing sample data to produce formatted raw sequencing sample data, wherein the formatted sequencing sample data is obtained using the formatted raw sequencing sample data to produce the sequencing variant sample data.

Clause 119. A computer program product comprising at least one non-transitory computer-readable storage medium storing software instructions that, when executed by an apparatus, cause the apparatus to: obtain subject sequencing variant sample data, wherein the subject sequencing variant sample data corresponds to sequenced sample data for a subject; generate, using a neural network trained to predict values of a genome map created from sample data and one or more reference genome maps, predicted values of a genome map for the subject based on the subject sequencing variant sample data; and determine, based on the predicted values, one or more predicted conditions for the subject.

Clause 120. The computer program product of clause 119, wherein the memory storing software instructions, when executed by the processor, further cause the apparatus to sequence a sample from the subject to produce raw sequenced sample data; and formatting the raw sequenced sample data to produce the sequenced sample data for the subject.

Clause 121. The computer program product of any of clauses 119 to 120, wherein the memory storing software instructions, when executed by the processor, further cause the apparatus to: receive reference data; identifying one or more values shared between the reference data and the sample data for the subject; and removing one or more of the one or more values shared between the reference data and the formatted sequencing sample data to yield the subject sequencing variant sample data for the subject.

Clause 122. The computer program product of any of clauses 119 to 121, wherein the sample is sequenced using one or more of whole exome sequencing or whole genome sequencing.

Clause 123. The computer program product of any of clauses 119 to 122, wherein the one or more predicted conditions comprise breast cancer, cardiovascular disease, type one diabetes, type two diabetes, schizophrenia, or a combination thereof.

Clause 124. The computer program product of any of clauses 119 to 123, wherein the one or more predicted conditions comprise a polygenic risk score determined for a particular condition for the subject, wherein one or more predicted risk scores for the particular condition are used to determine the polygenic risk score for the particular condition.

Clause 125. The computer program product of any of clauses 119 to 124, wherein the memory storing software instructions, when executed by the processor, further cause the apparatus to: obtain sequencing variant sample data, wherein the sequencing variant sample data is based on sequencing sample data and one or more reference genome maps; and train a neural network using the sequencing variant sample data, wherein training the neural network includes: altering values at one or more loci for one or more samples in the sequencing variant sample data to produce altered sequencing variant sample data, and using the altered sequencing variant sample data as input during training of the neural network such that the neural network is trained to predict values from unaltered sequencing variant sample data and the one or more reference genome maps.

Clause 126. The computer program product of any of clauses 119 to 125, wherein the software instructions, when executed by the processor and when training the neural network, further cause the apparatus to train one or more layers of the neural network by performing one or more training iterations, wherein each performing each training iteration further causes the apparatus to: alter one or more actual values of the sequencing variant sample data from the one or more loci and the one or more samples corresponding to the sequencing variant sample data; generate, using the neural network, one or more predicted values for the one or more loci and the one or more samples, and adjusting the neural network based on the one or more predicted values and the one or more actual values.

Clause 127. The computer program product of any of clauses 119 to 126, wherein the software instructions, when executed by the processor, further cause the apparatus to obtain phenotype data associated with the sequencing variant sample data; and train one or more layers of the neural network to predict the phenotype data.

Clause 128. The computer program product of any of clauses 119 to 127, wherein (i) the one or more layers are arranged into a hierarchy of attention encoder mechanisms, (ii) a level in the hierarchy is subdivided into one or more groups and (iii) for each group, using one or more parent tokens from another attention encoder mechanism associated with the group as an input token for an attention encoder mechanism at a subsequent level in the hierarchy.

Clause 129. The computer program product of any of clauses 119 to 128, wherein the software instructions, when executed by the processor, further cause the apparatus to append an input-type token to the one or more groups.

Clause 130. The computer program product of any of clauses 119 to 129, wherein the input-type token corresponds to an input type and the input type corresponds to one or more of a cell type, a tissue type, an exome sequence, a whole genome sequence, or a species.

Clause 131. The computer program product of any of clauses 119 to 130, wherein the sequencing variant sample data comprises variants obtained from whole exome sequencing.

Clause 132. The computer program product of any of clauses 119 to 131, wherein the sequencing variant sample data comprises variants obtained from whole genome sequencing.

Clause 133. The computer program product of any of clauses 119 to 132, wherein the phenotype data includes disease onset and control labels for one or more conditions.

Clause 134. The computer program product of any of clauses 119 to 133, wherein the one or more conditions comprise breast cancer, cardiovascular disease, type one diabetes, type two diabetes, schizophrenia, or a combination thereof.

Clause 135. The computer program product of any of clauses 119 to 134, wherein the software instructions, when executed by the processor and when altering the values, further cause the apparatus to mask a fraction of the values of the sequencing variant sample data.

Clause 136. The computer program product of any of clauses 119 to 135, wherein the software instructions, when executed by the processor and when altering the values, further cause the apparatus to mask a first fraction of the values of the sequencing variant sample data and replace a second fraction of the values of the sequencing variant sample data.

Clause 137. The computer program product of any of clauses 119 to 136, wherein the sequencing variant sample data is one-hot encoded.

Clause 138. The computer program product of any of clauses 119 to 137, wherein the software instructions, when executed by the processor and when obtaining the sequencing variant sample data, further cause the apparatus to: receive reference data and formatted sequencing sample data; identify one or more values shared between the reference data and the formatted sequencing sample data; and remove one or more of the one or more values shared between the reference data and the formatted sequencing sample data.

Clause 139. The computer program product of any of clauses 119 to 138, wherein the software instructions, when executed by the processor and when receiving the reference data, further cause the apparatus to: identify one or more reference datasets; and generate combined reference data by combining the one or more reference datasets with one or more epigenetic maps, wherein the reference data comprises the combined reference data.

Clause 140. The computer program product of any of clauses 119 to 139, wherein the one or more epigenetic maps comprises one or more deoxyribonucleic acid methylation maps.

Clause 141. The computer program product of any of clauses 119 to 140, wherein the software instructions, when executed by the processor and when receiving the formatted sequencing sample data, further cause the apparatus to access raw sequencing sample data; and format the raw sequencing sample data to produce formatted raw sequencing sample data, wherein the formatted sequencing sample data is obtained using the formatted raw sequencing sample data to produce the sequencing variant sample data.

What is claimed is:

1. A method for training a model to predict information based on sequencing sample data and one or more reference genome maps, the method comprising:
obtaining sequencing variant sample data, wherein the sequencing variant sample data is based on sequencing sample data and one or more reference genome maps; and
training a neural network using the sequencing variant sample data, wherein training the neural network includes:
altering values at one or more loci for one or more samples in the sequencing variant sample data to produce altered sequencing variant sample data, and
using the altered sequencing variant sample data as input during training of the neural network such that the neural network is trained to predict values from unaltered sequencing variant sample data and the one or more reference genome maps.

2-13. (canceled)

14. The method of claim 1, wherein obtaining the sequencing variant sample data further comprises:
receiving reference data and formatted sequencing sample data;
identifying one or more values shared between the reference data and the formatted sequencing sample data; and
removing one or more of the one or more values shared between the reference data and the formatted sequencing sample data.

15. The method of claim 14, wherein receiving the reference data further comprises:
identifying one or more reference datasets; and
generating combined reference data by combining the one or more reference datasets with one or more epigenetic maps, wherein the reference data comprises the combined reference data.

16. The method of claim 15, wherein the one or more epigenetic maps comprises one or more deoxyribonucleic acid methylation maps.

17. The method of claim 14, wherein receiving the formatted sequencing sample data further comprises:
accessing raw sequencing sample data; and
formatting the raw sequencing sample data to produce formatted raw sequencing sample data, wherein the formatted sequencing sample data is obtained using the formatted raw sequencing sample data to produce the sequencing variant sample data.

18-24. (canceled)

25. An apparatus for training a model to predict information based on sequencing sample data and one or more reference genome maps, the apparatus comprising a processor and a memory storing software instructions that, when executed by the processor, cause the apparatus to perform operations including:
obtaining sequencing variant sample data, wherein the sequencing variant sample data is based on sequencing sample data and one or more reference genome maps; and
training a neural network using the sequencing variant sample data, wherein training the neural network includes:
altering values at one or more loci for one or more samples in the sequencing variant sample data to produce altered sequencing variant sample data, and
using the altered sequencing variant sample data as input during training of the neural network such that the neural network is trained to predict values from unaltered sequencing variant sample data and the one or more reference genome maps.

26. (canceled)

27. A method for generating, using a trained model, a prediction regarding a genome map of a subject based sequenced sample data for the subject, the method comprising:
obtaining subject sequencing variant sample data, wherein the subject sequencing variant sample data corresponds to sequenced sample data for a subject;
generating, using a neural network trained to predict values of a genome map created from sample data and one or more reference genome maps, predicted values of a genome map for the subject based on the subject sequencing variant sample data; and
determining, based on the predicted values, one or more predicted conditions for the subject.

28-51. (canceled)

52. The method of claim 1, further comprising:
obtaining phenotype data associated with the sequencing variant sample data; and
training one or more layers of the neural network to predict the phenotype data.

53. The method of claim 52, wherein the phenotype data includes disease onset and control labels for one or more conditions.

54. The method of claim 53, wherein the one or more conditions comprise breast cancer, cardiovascular disease, type one diabetes, type two diabetes, schizophrenia, or a combination thereof.

55. The method of claim 52, further comprising:
obtaining subject sequencing variant sample data, wherein the subject sequencing variant sample data corresponds to sequenced sample data for a subject; and
generating, using the neural network, predicted values of a genome map for the subject based on the subject sequencing variant sample data.

56. The method of claim 55, further comprising determining one or more predicted conditions for the subject.

57. The method of claim 56, wherein the one or more predicted conditions comprise a polygenic risk score determined for a particular condition for the subject, wherein one or more predicted risk scores for the particular condition are used to determine the polygenic risk score for the particular condition.

58. The method of claim 52, wherein the sequencing variant sample data comprises variants obtained from whole exome sequencing or whole genome sequencing.

59. The method of claim 1, wherein training the neural network comprises training one or more layers of the neural network by performing one or more training iterations, each training iteration including:
altering one or more actual values of the sequencing variant sample data from the one or more loci and the one or more samples corresponding to the sequencing variant sample data;
generating, using the neural network, one or more predicted values for the one or more loci and the one or more samples, and
adjusting the neural network based on the one or more predicted values and the one or more actual values.

60. The method of claim 59, wherein (i) the one or more layers are arranged into a hierarchy of attention encoder mechanisms, (ii) a level in the hierarchy is subdivided into one or more groups and (iii) for each group, using one or more parent tokens from another attention encoder mechanism associated with the group as an input token for an attention encoder mechanism at a subsequent level in the hierarchy.

61. The method of claim 60, further comprising appending an input-type token to the one or more groups.

62. The method of claim 61, wherein the input-type token corresponds to an input type and the input type corresponds to one or more of a cell type, a tissue type, an exome sequence, a whole genome sequence, or a species.

63. The method of claim 1, wherein altering the values further comprises masking or replacing a fraction of the values of the sequencing variant sample data.

64. The method of claim 1, wherein the sequencing variant sample data is one-hot encoded.
